(19) United States
(12) Patent Application Publication
(10) Pub. No.: US 2025/0248664 A1
(43) Pub. Date: Aug. 7, 2025

(54) IMAGE DIAGNOSTIC SYSTEM AND METHOD

(71) Applicant: TERUMO KABUSHIKI KAISHA, Tokyo (JP)

(72) Inventor: Yuki SAKAGUCHI, Fujisawa Kanagawa (JP)

(21) Appl. No.: 19/094,717

(22) Filed: Mar. 28, 2025

Related U.S. Application Data
(63) Continuation of application No. PCT/JP2023/035480, filed on Sep. 28, 2023.

(30) Foreign Application Priority Data
Sep. 30, 2022 (JP) 2022-158097

(51) Int. Cl. A61B 5/00; A61B 6/00; A61B 6/46; A61B 6/50; A61B 8/00; A61B 8/08; A61B 8/12

(52) U.S. Cl. CPC A61B 5/743; A61B 5/0035; A61B 5/0066; A61B 5/0084; A61B 5/7267; A61B 6/463; A61B 6/481; A61B 6/504; A61B 6/5247; A61B 8/0891; A61B 8/12; A61B 8/463; A61B 8/5261

(57) ABSTRACT

An image diagnostic system includes a catheter insertable into a blood vessel, a memory that stores a program, and a processor configured to execute the program to: control the catheter to acquire a tomographic image of a blood vessel, input the acquired image into a computer model to generate information that indicates a plurality of predetermined regions of the blood vessel in the image, the information further indicating for each of the predetermined regions whether it overlaps another region, the computer model having been trained with a plurality of tomographic images of blood vessels and a plurality of information each specifying the predetermined regions that can overlap in a corresponding one of the tomograph images, and using the generated information, determine the predetermined regions of the blood vessel in the acquired image, and output information indicating the determined regions.

### CROSS-REFERENCE TO RELATED APPLICATION

This application is a continuation of International Patent Application No. PCT/JP2023/035480 filed Sep. 28, 2023, which is based upon and claims the benefit of priority from Japanese Patent Application No. 2022-158097, filed Sep. 30, 2022, the entire contents of which are incorporated herein by reference.

### BACKGROUND

### Technical Field

Embodiments described herein relate to an image diagnostic system and method.

### Related Art

A medical image of a blood vessel such as an ultrasonic tomographic image is generated by an intravascular ultrasound (IVUS) method using a catheter for performing an ultrasonic inspection of the blood vessel. Meanwhile, for the purpose of assisting a doctor in making a diagnosis, a technology of adding information to a medical image by image processing or machine learning has been developed. In such a technology, feature data indicating a luminal wall and a stent in a blood vessel can be individually extracted from the blood vessel image.

However, in the conventional technique, it is difficult to separate and extract a plurality of overlapping regions, such as a region indicating the inside of the stent, a region indicating the lumen of the blood vessel, and a region indicating the inside of the external elastic membrane.

### SUMMARY

In one embodiment, an image diagnostic system comprises a catheter insertable into a blood vessel; a memory that stores a program; and a processor configured to execute the program to: control the catheter to acquire a tomographic image of a blood vessel, input the acquired image into a computer model to generate information that indicates a plurality of predetermined regions of the blood vessel in the image, the information further indicating for each of the predetermined regions whether it overlaps another region, the computer model having been trained with a plurality of tomographic images of blood vessels and a plurality of information each specifying the predetermined regions that can overlap in a corresponding one of the tomograph images, and using the generated information, determine the predetermined regions of the blood vessel in the acquired image, and output information indicating the determined regions.

Based on the configuration described above, it is possible to provide an image diagnostic system and method capable of identifying a plurality of overlapping regions from a tomographic image of a blood vessel.

### BRIEF DESCRIPTION OF DRAWINGS

FIG. 1 is a schematic diagram illustrating an image diagnosis system according to a first embodiment.

FIG. 2 is a schematic diagram illustrating an image diagnosis catheter.

FIG. 3 is an explanatory diagram illustrating a cross section of a blood vessel through which a sensor unit is inserted.

FIG. 4A is an explanatory diagram for explaining a tomographic image.

FIG. 4B is an explanatory diagram for explaining a tomographic image.

FIG. 5 is a block diagram illustrating a configuration of an image processing apparatus.

FIG. 6 is a diagram for explaining a conventional annotation method.

FIG. 7 is a diagram for explaining an annotation method according to the first embodiment.

FIG. 8 is a diagram for explaining an annotation method according to the first embodiment.

FIG. 9 is a diagram for explaining an annotation method according to the first embodiment.

FIG. 10 is a diagram for explaining a work environment of annotation.

FIG. 11 is an explanatory diagram for explaining a configuration of a learning model.

FIG. 12 is a flowchart for explaining an annotation execution procedure according to the first embodiment.

FIG. 13 is a flowchart for explaining a learning model generation procedure.

FIG. 14 is a flowchart for explaining a region recognition procedure by a learning model.

FIG. 15 is a diagram for explaining configurations of a first learning model and a second learning model according to a second embodiment.

### DETAILED DESCRIPTION

Hereinafter, embodiments of the present invention will be described in detail with reference to the drawings.

### First Embodiment

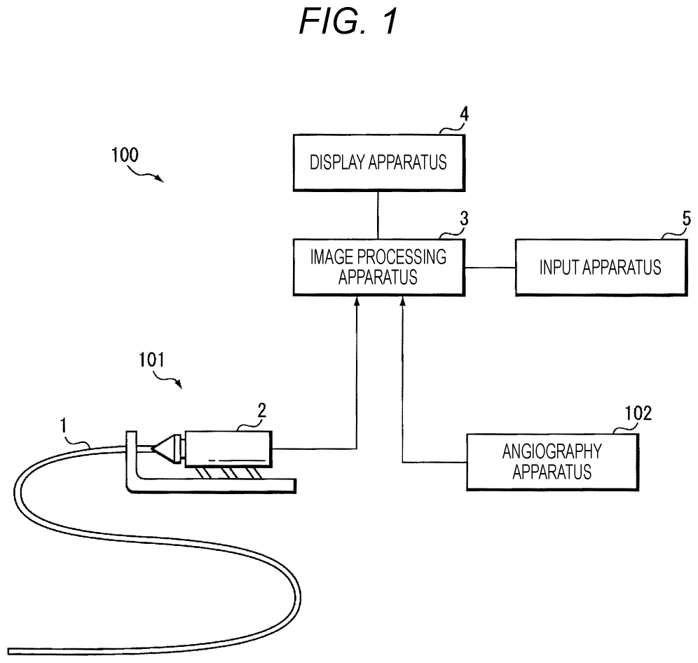

FIG. 1 is a schematic diagram illustrating an image diagnosis system 100 according to a first embodiment. In the present embodiment, the image diagnosis system 100 includes a dual type catheter having functions of both intravascular ultrasound diagnosis method (IVUS) and optical coherence tomography (OCT) will be described. In the dual type catheter, a mode of acquiring an ultrasonic tomographic image only by IVUS, a mode of acquiring an optical coherence tomographic image only by OCT, and a mode of acquiring both tomographic images by IVUS and OCT are provided, and these modes can be switched and used. Hereinafter, the ultrasonic tomographic image and the optical coherence tomographic image are also referred to as an IVUS image and an OCT image, respectively. The IVUS image and the OCT image are examples of tomographic images of a blood vessel, and in a case where it is not necessary to distinguish and describe the IVUS image and the OCT image, they are also simply described as tomographic images.

The image diagnosis system 100 according to the embodiment includes an intravascular inspection apparatus 101, an angiography apparatus 102, an image processing apparatus 3, a display apparatus 4, and an input apparatus 5. The intravascular inspection apparatus 101 includes an image diagnosis catheter 1 and a motor drive unit (MDU) 2. The image diagnosis catheter 1 is connected to the image processing apparatus 3 via the MDU 2. The display apparatus 4 and the input apparatus 5 are connected to the image processing apparatus 3. The display apparatus 4 is, for example, a liquid crystal display, an organic electro-luminescence (EL) display, or the like, and the input apparatus 5 is, for example, a keyboard, a mouse, a touch panel, a microphone, or the like. The input apparatus 5 and the image processing apparatus 3 may be integrated into a single apparatus. Furthermore, the input apparatus 5 may be a sensor that receives a gesture input, a line-of-sight input, or the like.

The angiography apparatus 102 is connected to the image processing apparatus 3. The angiography apparatus 102 images a blood vessel from outside a living body of a patient using X-rays while injecting a contrast agent into the blood vessel of the patient to obtain an angiographic image that is a fluoroscopic image of the blood vessel. The angiography apparatus 102 includes an X-ray source and an X-ray sensor, and images an X-ray fluoroscopic image of the patient by the X-ray sensor receiving X-rays emitted from the X-ray source. Note that the image diagnosis catheter 1 has a marker that does not transmit X-rays, and the position of the image diagnosis catheter 1 (i.e., the marker) is visualized in the angiographic image. The angiography apparatus 102 outputs the angiographic image obtained by imaging to the image processing apparatus 3, and causes the display apparatus 4 to display the angiographic image via the image processing apparatus 3. Note that the display apparatus 4 displays the angiographic image and the tomographic image imaged using the image diagnosis catheter 1.

Note that, in the present embodiment, the image processing apparatus 3 is connected to the angiography apparatus 102 that images two-dimensional angiographic images. However, the present invention is not limited to the angiography apparatus 102 as long as it is an apparatus that images a luminal organ of a patient and the image diagnosis catheter 1 from a plurality of directions outside the living body.

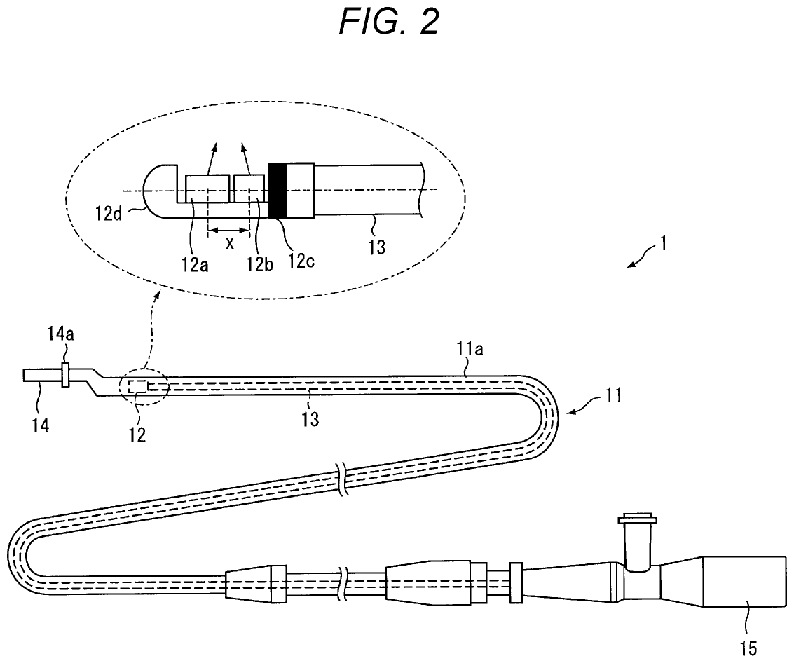

FIG. 2 is a schematic diagram illustrating the image diagnosis catheter 1. Note that a region indicated by a one-dot chain line on an upper side in FIG. 2 is an enlarged view of a region indicated by a one-dot chain line on a lower side. The image diagnosis catheter 1 includes a probe 11 and a connector portion 15 disposed at an end of the probe 11. The probe 11 is connected to the MDU 2 via the connector portion 15. In the following description, a side far from the connector portion 15 of the image diagnosis catheter 1 will be referred to as a distal end side, and a side of the connector portion 15 will be referred to as a proximal end side. The probe 11 includes a catheter sheath 11a, and a guide wire insertion portion 14 through which a guide wire can be inserted is provided at a distal portion thereof. The guide wire insertion portion 14 forms a guide wire lumen, receives a guide wire previously inserted into a blood vessel, and guides the probe 11 to an affected part by the guide wire. The catheter sheath 11a forms a tube portion continuous from a connection portion with the guide wire insertion portion 14 to a connection portion with the connector portion 15. A shaft 13 is inserted into the catheter sheath 11a, and a sensor unit 12 is connected to a distal end side of the shaft 13.

The sensor unit 12 includes a housing 12d, and a distal end side of the housing 12d is formed in a hemispherical shape in order to suppress friction and catching with an inner surface of the catheter sheath 11a. In the housing 12d, an ultrasound transmitter and receiver 12a (hereinafter referred to as an IVUS sensor 12a) that transmits ultrasonic waves into a blood vessel and receives reflected waves from the blood vessel and an optical transmitter and receiver 12b (hereinafter referred to as an OCT sensor 12b) that transmits near-infrared light into the blood vessel and receives reflected light from the inside of the blood vessel are disposed. In the example illustrated in FIG. 2, the IVUS sensor 12a is provided on the distal end side of the probe 11, the OCT sensor 12b is provided on the proximal end side thereof, and the IVUS sensor 12a and the OCT sensor 12b are arranged apart from each other by a distance x along the axial direction on a central axis (on a two-dot chain line in FIG. 2) of the shaft 13. In the image diagnosis catheter 1, the IVUS sensor 12a and the OCT sensor 12b are attached such that a direction that is approximately 90 degrees with respect to the axial direction of the shaft 13 (i.e., the radial direction of the shaft 13) is set as a transmission/reception direction of an ultrasonic wave or near-infrared light. Note that the IVUS sensor 12a and the OCT sensor 12b are desirably attached slightly shifted from the radial direction so as not to receive a reflected wave or reflected light on the inner surface of the catheter sheath 11a. In the present embodiment, for example, as indicated by an arrow in FIG. 2, the IVUS sensor 12a is attached with a direction inclined to the proximal end side with respect to a radial direction as an irradiation direction of the ultrasonic wave, and the OCT sensor 12b is attached with a direction inclined to the distal end side with respect to the radial direction as an irradiation direction of the near-infrared light.

An electric signal cable (not illustrated) connected to the IVUS sensor 12a and an optical fiber cable (not illustrated) connected to the OCT sensor 12b are inserted into the shaft 13. The probe 11 is inserted into the blood vessel from the distal end side. The sensor unit 12 and the shaft 13 can move forward or rearward inside the catheter sheath 11a and can rotate in a circumferential direction. The sensor unit 12 and the shaft 13 rotate about the central axis of the shaft 13 as a rotation axis. In the image diagnosis system 100, by using an imaging core including the sensor unit 12 and the shaft 13, a state inside the blood vessel is measured by an ultrasonic tomographic image (IVUS image) captured from the inside of the blood vessel or an optical coherence tomographic image (i.e., an OCT image) captured from the inside of the blood vessel.

The MDU 2 is a drive apparatus to which the probe 11 is detachably attached by the connector portion 15, and controls the operation of the image diagnosis catheter 1 inserted into the blood vessel by driving a built-in motor according to an operation of a medical worker. For example, the MDU 2 performs a pull-back operation of rotating the sensor unit 12 and the shaft 13 inserted into the probe 11 in the circumferential direction while pulling the sensor unit 12 and the shaft 13 toward the MDU 2 side at a constant speed. The sensor unit 12 continuously scans the inside of the blood vessel at predetermined time intervals while moving and rotating from the distal end side to the proximal end side by the pull-back operation and continuously captures a plurality of transverse tomographic images substantially perpendicular to the probe 11 at predetermined intervals. The MDU 2 outputs reflected wave data of an ultrasonic wave received by the IVUS sensor 12a and reflected light data received by the OCT sensor 12b to the image processing apparatus 3.

The image processing apparatus 3 acquires a signal data set which is the reflected wave data of the ultrasonic wave received by the IVUS sensor 12a and a signal data set which is reflected light data received by the OCT sensor 12b via the MDU 2. The image processing apparatus 3 generates ultrasonic line data from a signal data set of the ultrasonic waves, and constructs an ultrasonic tomographic image (i.e., an IVUS image) obtained by imaging a transverse section of the blood vessel based on the generated ultrasonic line data. In addition, the image processing apparatus 3 generates optical line data from the signal data set of the reflected light, and constructs an optical coherence tomographic image (i.e., an OCT image) obtained by imaging a transverse section of the blood vessel based on the generated optical line data. Here, the signal data set acquired by the IVUS sensor 12a and the OCT sensor 12b and the tomographic image constructed from the signal data set will be described.

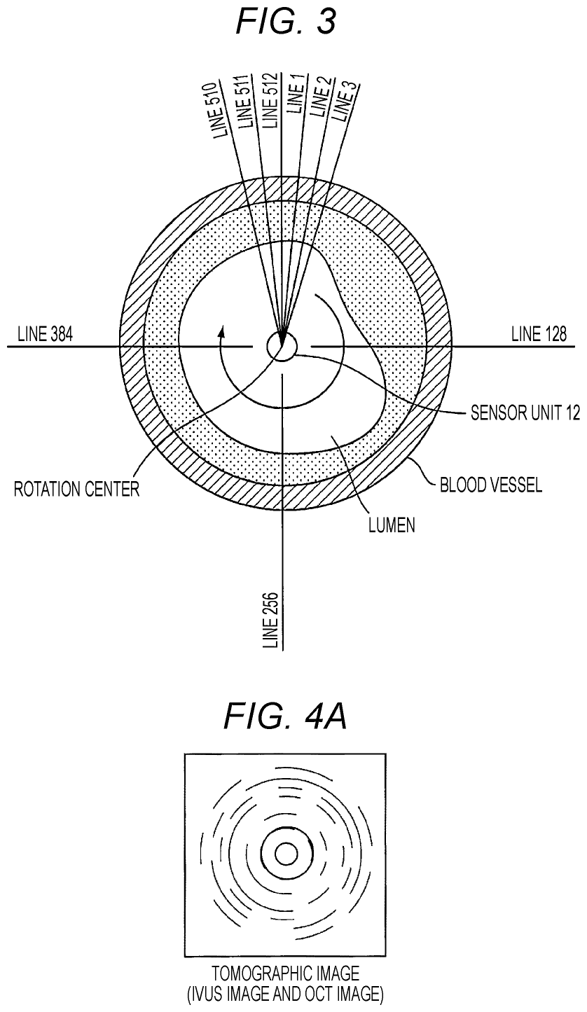

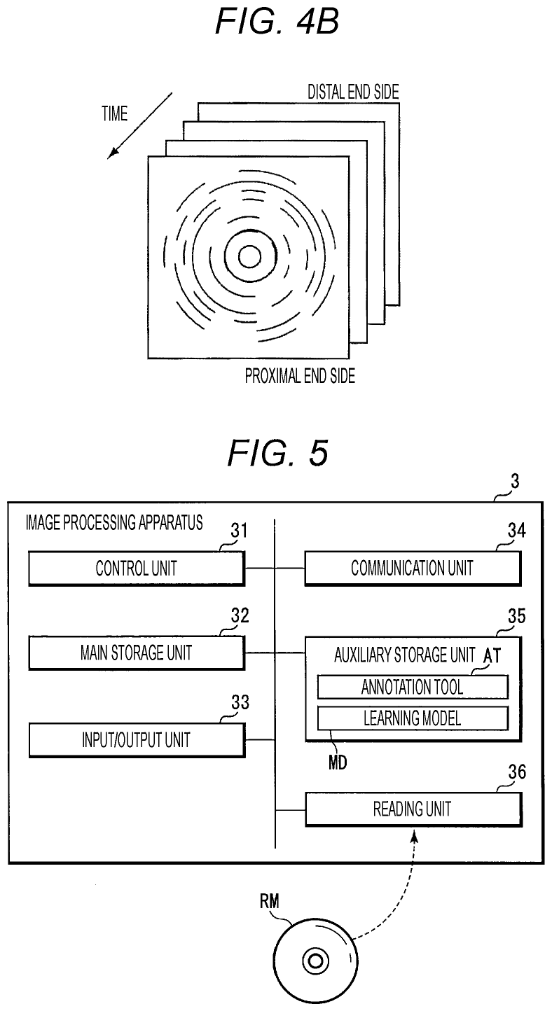

FIG. 3 is a diagram for explaining a cross section of a blood vessel through which the sensor unit 12 is inserted, and FIGS. 4A and 4B are diagrams for explaining tomographic images. First, with reference to FIG. 3, operations of the IVUS sensor 12a and the OCT sensor 12b in the blood vessel, and signal data sets (e.g., ultrasonic line data and optical line data) acquired by the IVUS sensor 12a and the OCT sensor 12b will be described. When the imaging of the tomographic image is started in a state where the imaging core is inserted into the blood vessel, the imaging core rotates about a central axis of the shaft 13 as a rotation center in a direction indicated by an arrow. At this time, the IVUS sensor 12a transmits and receives an ultrasonic wave at each rotation angle. Lines 1, 2, . . . 512 indicate transmission/reception directions of ultrasonic waves at each rotation angle. In the present embodiment, the IVUS sensor 12a intermittently transmits and receives ultrasonic waves 512 times while rotating 360 degrees (i.e., 1 rotation) in the blood vessel. Since the IVUS sensor 12a acquires data of one line in the transmission/reception direction by transmitting and receiving an ultrasonic wave once, it is possible to obtain 512 pieces of ultrasonic line data radially extending from the rotation center during one rotation. The 512 pieces of ultrasonic line data are dense in the vicinity of the rotation center, but become sparse with distance from the rotation center. Therefore, the image processing apparatus 3 can generate a two-dimensional ultrasonic tomographic image (i.e., an IVUS image) as illustrated in FIG. 4A by generating pixels in an empty space of each line by known interpolation processing.

Similarly, the OCT sensor 12b also transmits and receives the measurement light at each rotation angle. Since the OCT sensor 12b also transmits and receives the measurement light 512 times while rotating 360 degrees in the blood vessel, it is possible to obtain 512 pieces of optical line data radially extending from the rotation center during one rotation. Moreover, for the optical line data, the image processing apparatus 3 can generate a two-dimensional optical coherence tomographic image (i.e., an OCT image) similar to the IVUS image illustrated in FIG. 4A by generating pixels in an empty space of each line by known interpolation processing. That is, the image processing apparatus 3 generates optical line data based on interference light generated by causing reflected light and, for example, reference light obtained by separating light from a light source in the image processing apparatus 3 to interfere with each other, and constructs an optical coherence tomographic image obtained by imaging the transverse section of the blood vessel based on the generated optical line data.

The two-dimensional tomographic image generated from the 512 pieces of line data in this manner is referred to as an IVUS image or an OCT image of one frame. Note that, since the sensor unit 12 scans while moving in the blood vessel, an IVUS image or an OCT image of one frame is acquired at each position rotated once within a movement range. That is, since the IVUS image or the OCT image of one frame is acquired at each position from the distal end side to the proximal end side of the probe 11 in the movement range, as illustrated in FIG. 4B, the IVUS image or the OCT image of a plurality of frames is acquired within the movement range.

The image diagnosis catheter 1 has a marker that does not transmit X-rays in order to confirm a positional relationship between the IVUS image obtained by the IVUS sensor 12a or the OCT image obtained by the OCT sensor 12b and the angiographic image obtained by the angiography apparatus 102. In the example illustrated in FIG. 2, a marker 14a is provided at the distal portion of the catheter sheath 11a, for example, the guide wire insertion portion 14, and a marker 12c is provided on the shaft 13 side of the sensor unit 12. When the image diagnosis catheter 1 configured as described above is imaged with X-rays, an angiographic image in which the markers 14a and 12c are visualized is obtained. The positions of the markers 14a and 12c are mere examples, and the marker 12c may be provided on the shaft 13 instead of the sensor unit 12, and the marker 14a may be provided at a portion other than the distal portion of the catheter sheath 11a.

FIG. 5 is a block diagram illustrating a configuration of the image processing apparatus 3. The image processing apparatus 3 includes a control unit 31, a main storage unit 32, an input/output unit 33, a communication unit 34, an auxiliary storage unit 35, and a reading unit 36. The image processing apparatus 3 is not limited to a single apparatus, and may be formed by a plurality of apparatuses. In addition, the image processing apparatus 3 may be a server client system, a cloud server, or virtual machine virtually constructed by software. In the following description, it is assumed that the image processing apparatus 3 is a single apparatus.

The control unit 31 includes one or more processors such as central processing units (CPU), micro processing units (MPU), graphics processing units (GPU), general purpose computing on graphics processing units (GPGPU), tensor processing units (TPU), and field programmable gate arrays (FPGA). The control unit 31 is connected to each hardware unit constituting the image processing apparatus 3 via a bus.

The main storage unit 32, which is a temporary memory area such as a static random access memory (SRAM), a dynamic random access memory (DRAM), or a flash memory, temporarily stores data necessary for the control unit 31 to execute arithmetic processing.

The input/output unit 33 includes an interface circuit that connects external apparatuses such as the intravascular inspection apparatus 101, the angiography apparatus 102, the display apparatus 4, and the input apparatus 5. The control unit 31 acquires an IVUS image and an OCT image from the intravascular inspection apparatus 101 via the input/output unit 33, and acquires an angiographic image from the angiography apparatus 102. In addition, the control unit 31 outputs a medical image signal of an IVUS image, an OCT image, or an angiographic image to the display apparatus 4 via the input/output unit 33, thereby displaying the medical image on the display apparatus 4. Furthermore, the control unit 31 receives information input to the input apparatus 5 via the input/output unit 33.

The communication unit 34 includes, for example, a communication interface circuit conforming to a communication standard such as 4G, 5G, or WiFi. The image processing apparatus 3 communicates with an external server such as a cloud server connected to an external network such as the Internet via the communication unit 34. The control unit 31 may access an external server via the communication unit 34 and refer to various data stored in a storage of the external server. Furthermore, the control unit 31 may cooperatively perform the processing in the present embodiment by performing, for example, inter-process communication with the external server.

The auxiliary storage unit 35 is a storage device such as a hard disk drive (HDD) or a solid state drive (SSD). The auxiliary storage unit 35 stores a computer program executed by the control unit 31 and various data necessary for processing of the control unit 31. Note that the auxiliary storage unit 35 may be an external storage device connected to the image processing apparatus 3. The computer program executed by the control unit 31 may be written in the auxiliary storage unit 35 at the manufacturing stage of the image processing apparatus 3, or the computer program distributed by a remote server apparatus may be acquired by the image processing apparatus 3 through communication and stored in the auxiliary storage unit 35. The computer program may be readably recorded in a recording medium RM such as a magnetic disk, an optical disk, or a semiconductor memory, or may be read from the recording medium RM by the reading unit 36 and stored in the auxiliary storage unit 35.

In addition, the auxiliary storage unit 35 may store a computer learning model MD used for processing of identifying a plurality of regions to be recognized from a tomographic image of a blood vessel including an IVUS image and an OCT image. When a tomographic image of a blood vessel is input, the learning model MD is trained to output information for identifying a plurality of regions to be recognized. Here, the regions to be recognized by the learning model MD includes at least two of a region indicating the inside of the stent placed in the blood vessel, a region indicating the lumen of the blood vessel, and a region indicating the inside of the external elastic membrane constituting the blood vessel. In addition, the regions to be recognized may include a region surrounded by the adventitia of the blood vessel (hereinafter also referred to as a blood vessel region). Furthermore, for each of the main trunk and the side branch of the blood vessel, a region indicating the lumen and a region indicating the inside of the external elastic membrane (or a region surrounded by the adventitia) may be recognized.

In addition, the regions to be recognized may further include at least one of a region where a plaque has occurred (hereinafter also referred to as a plaque region), a region where a thrombus has occurred (hereinafter also referred to as a thrombus region), and a region where a hematoma has occurred (hereinafter also referred to as a hematoma region). For the plaque region, it may be configured to recognize each region by distinguishing between calcified plaque, fibrous plaque, and lipid plaque. In addition, the regions to be recognized may also include regions such as dissection, perforation, or the like caused by vascular complications. Further, the regions to be recognized may include regions of extravascular structures such as veins and epicardium. Furthermore, the regions to be recognized may include a region where a device such as a guide wire, a guiding catheter, or a stent exists (hereinafter also referred to as a device region). Furthermore, the regions to be recognized may include image artifacts that occur at the time of imaging or at the time of image reconstruction due to scattered rays or noise. In addition, the regions to be recognized may be separately set on the IVUS image and the OCT image. For example, it is also possible to set a region indicating the lumen in the IVUS image and a region indicating the lumen in the OCT image as different regions. Furthermore, even in the same IVUS image, for example, a region indicating a lumen in an IVUS image generated using 40 MHz ultrasound and a region indicating a lumen in an IVUS image generated using 60 MHz ultrasound may be set as different regions.

In the present embodiment, the annotation for a large number of tomographic images is performed in the training phase before the recognition processing by the learning model MD is started. Specifically, in the image processing apparatus 3, the annotation tool AT is activated, and the annotation (i.e., designation of the region) is received in the work environment provided by the tool. Note that the annotation tool AT is one of computer programs installed in the image processing apparatus 3. Hereinafter, the annotation method of the present embodiment will be described while being compared with the conventional technique.

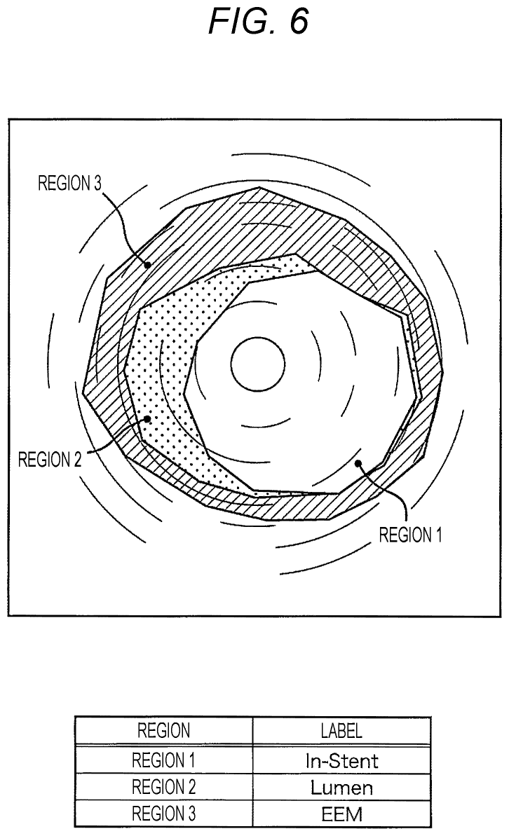

FIG. 6 is a diagram for explaining a conventional annotation method. As a method for simultaneously detecting a vascular lumen, a stent, and a vascular contour from a tomographic image of a blood vessel, a single label segmentation task is known. Here, with respect to the vascular lumen, a region surrounded by the inner membrane of the blood vessel (i.e., Lumen region) is a detection target. The stent appears as a plurality of minute regions corresponding to the position of the strut in the tomographic image. For the stent, for example, a boundary is set at the position of the strut, and an inner region (i.e., In-Stent region) is detected as a detection target. As for the vascular contour, since the surface layer of the adventitia of the blood vessel cannot be specified by the IVUS image, a boundary is alternatively set at the position of the external elastic membrane (or External Elastic Lamina), and a region inside the external elastic membrane (i.e., EEM region) is often set as a detection target. The external elastic membrane is a thin layer formed mainly of elastic tissue and separating the media and adventitia of the blood vessel.

When the annotation is performed on the captured tomographic image, in the single label segmentation task, the Lumen region, the In-Stent region, and the EEM region are separated from the tomographic image, and different labels are given to the separated regions. The example of FIG. 6 illustrates a state in which the Lumen region, the In-Stent region, and the EEM region are separated from the tomographic image. Here, the innermost white region (region 1) is a region separated as an In-Stent region. The region 1 is labeled “In-Stent”. A crescent region (region 2) indicated by dots is a region separated as a Lumen region. The region 2 is labeled “Lumen”. A donut-shaped region (region 3) indicated by hatching is a region separated as an EEM region. The region 3 is labeled “EEM”.

In the single label segmentation task, a learning model is constructed using the segmentation image to which one label is given for each region as described above as training data, and a vascular lumen (i.e., Lumen region), a stent (i.e., In-Stent region), and a vascular contour (i.e., EEM region) are simultaneously detected from a newly imaged tomographic image using the constructed learning model.

As a result of training by the conventional technique as described above, there is a possibility that a learning model is constructed so as to recognize a donut-shaped region as an EEM region although the EEM region is not actually a donut-shaped region. In addition, there is a possibility that the learning model is constructed so as to recognize the crescent region as the Lumen region although the Lumen region is not actually the crescent region. At this time, when the shapes of the EEM region and the Lumen region change due to narrowing or occlusion of the vascular lumen, there is a possibility that these regions cannot be correctly recognized.

In addition, when the stent is placed in close contact with the vascular lumen, the region having the label “Lumen” disappears, and there is a possibility that the vascular lumen cannot be detected. Furthermore, as time elapses after placing the stent, neointima may occur inside the stent. In this case, the Lumen region and the In-Stent region are reversed (that is, the In-Stent region exists outside the Lumen region), and erroneous determination may increase. In addition, there is also a concern that a rule becomes complicated in order to make a correct determination.

In the present embodiment, a plurality of regions can be simultaneously detected as a multi-label segmentation task as described below.

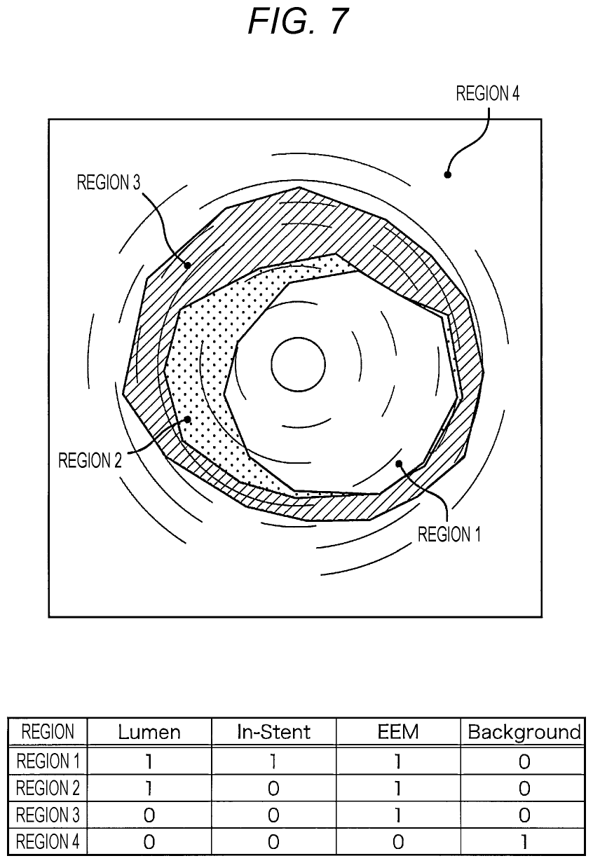

FIGS. 7 to 9 are diagrams for explaining an annotation method in the present embodiment. The tomographic image illustrated in FIG. 7 is similar to FIG. 6, and illustrates a state in which a stent is placed in a vascular lumen. The innermost white region (region 1) is an In-Stent region, but has an overlap with Lumen and EEM regions, so that not only the label “In-Stent” but also the labels “Lumen” and “EEM” are given. A crescent shaped region (region 2) existing outside the region 1 is a Lumen region, but since it has an overlap with the EEM region, not only the “Lumen” label but also the “EEM” label is given. A donut-shaped region (region 3) existing outside the region 2 is the EEM region that does not overlap with any other regions, so that only the label “EEM” is given. The table illustrated on the lower side of the tomographic image of FIG. 7 illustrates a label assignment status for each region. In this table, “1” indicates that a label is given, and “0” indicates that no label is given. Note that the region 4 indicates a background region existing outside the EEM region, and “Background” indicating the background is assigned as a label.

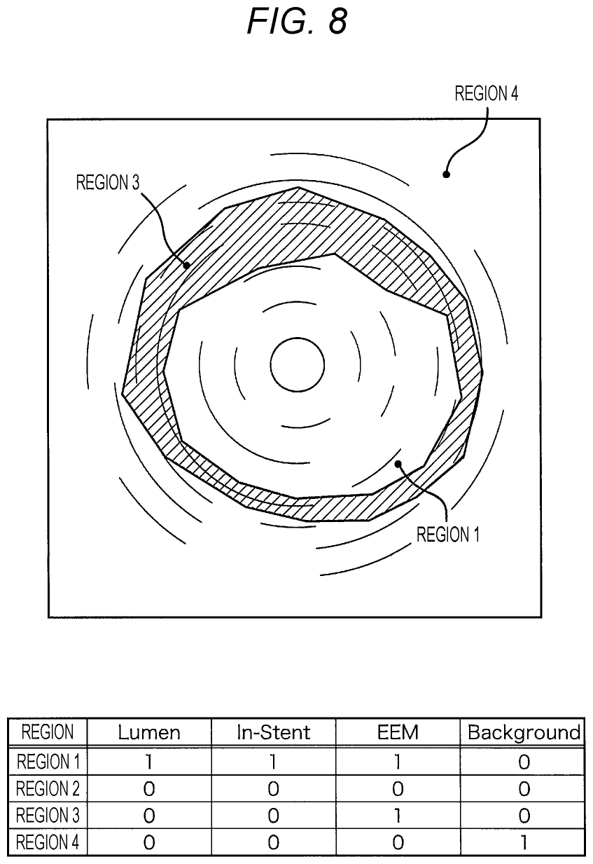

The tomographic image illustrated in FIG. 8 illustrates a state in which a stent is placed in close contact with a vascular lumen. In this case, since the innermost white region 1 is an In-Stent region but has an overlap with Lumen and EEM regions, not only the label “In-Stent” but also the labels “Lumen” and “EEM” are given. A donut- shaped region (region 3) existing outside the region 1 is an EEM region that does not overlap with other regions, so that only the label “EEM” is given. The region 4 indicates a background region existing outside the EEM region, and “Background” indicating the background is assigned as a label.

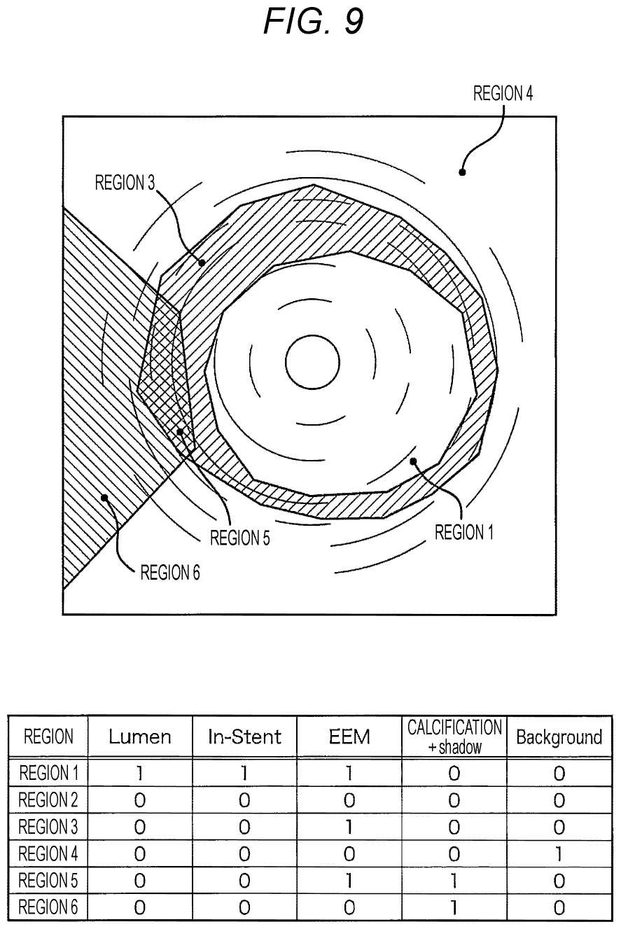

The tomographic image illustrated in FIG. 9 illustrates a state in which a stent is placed in close contact with a vascular lumen, and calcification is partially generated. The region 1, the region 3, and the region 4 are similar to those in FIG. 8. The region 5 illustrates a calcified region (i.e., a region where calcified plaque has occurred) inside EEM and is labeled “EEM” and “calcification+shadow”. In addition, the region 6 illustrates a calcified region outside EEM and is labeled “calcification+shadow” and “Background”. In the present embodiment, a label of “calcification +shadow” is given, but an individual label may be given such that a label of “calcification” is given to a calcified region and a label of “shadow” is given to a shadow region. Although FIG. 9 illustrates an example of a calcified region, the same applies to each region of a plaque region including a fibrous plaque or a lipid plaque, a thrombus region, a hematoma region, and a device region, and it is preferable to assign an individual label according to each region. Furthermore, in the present embodiment, the label is applied to the In-Stent region, but in a case where each strut and the shadow/shade portion behind the strut are detected as the detection target, a label (for example, “strut+shadow” or “strut” and “shadow”) for identifying these regions may be applied. In the OCT, the stent and the strut are clearly drawn, and the strut region (+shadow) region may be distinguishably recognized together with the In-Stent region in order to calculate the malapposition and the expansion rate. The image processing apparatus 3 according to the present embodiment receives the annotation for the tomographic image through the working environment provided by the annotation tool AT.

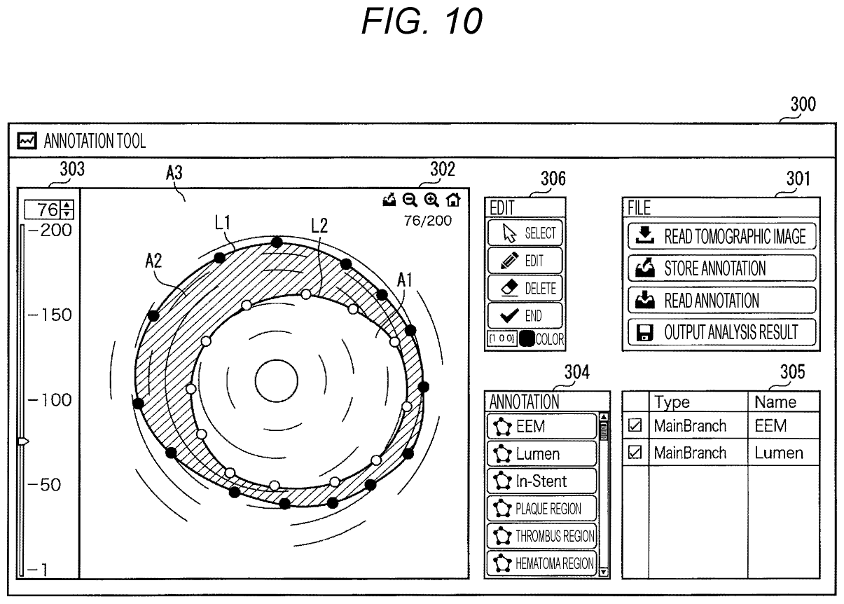

FIG. 10 is a diagram for explaining a work environment of annotation. When the annotation tool AT is activated, the image processing apparatus 3 displays a work screen 300 as illustrated in FIG. 10 on the display apparatus 4. The work screen 300 includes one or more graphical user interface (GUI) components including a file selection tool 301, an image display field 302, a frame designation tool 303, a region designation tool 304, a segment display field 305, an editing tool 306, and the like, and receives various operations through the input apparatus 5.

The file selection tool 301 is a tool for receiving selection operations of various files, and includes software buttons for reading a tomographic image, storing annotation data, reading annotation data, and outputting an analysis result. When the tomographic image is read by the file selection tool 301, the read tomographic image is displayed in the image display field 302. The tomographic image generally includes a plurality of frames. The frame designation tool 303 includes an input box and a slider for designating a frame, and is used to designate a frame of a tomographic image to be displayed in the image display field 302. The example of FIG. 10 illustrates a state in which the 76th frame among 200 frames is designated.

The region designation tool 304 is a tool for receiving designation of a region for the tomographic image displayed in the image display field 302, and includes software buttons corresponding to the respective labels. In the example of FIG. 10, software buttons corresponding to the labels “EEM”, “Lumen”, “In-Stent”, “plaque region”, “thrombus region”, and “hematoma region” are illustrated. The number of software buttons and the type of labels are not limited to the above, and can be arbitrarily set by the user.

When designating the EEM region using the region designation tool 304, the user selects the software button labeled “EEM” and plots a plurality of points so as to surround the EEM region on the image display field 302. The same applies to the case of designating other regions. The control unit 31 of the image processing apparatus 3 derives a smooth closed curve by spline interpolation or the like based on a plurality of points plotted by the user, and draws the derived closed curve in the image display field 302. The interior of the closed curve is drawn in a preset color or a color set by the user.

The example of FIG. 10 illustrates a state in which the EEM region is designated by a closed curve L1 based on a plurality of points indicated by black circles, and the Lumen region is designated by a closed curve L2 based on a plurality of points indicated by white circles. In this example, the image display field 302 is divided into three regions of a region A1 inside the closed curve L2, a region A2 between the closed curve L1 and the closed curve L2, and a region A3 outside the closed curve L1. Since the region A1 is a region in which the EEM region and the Lumen region overlap, the control unit 31 assigns labels “EEM” and “Lumen” to the region A1. Since the region A2 is a region where the EEM region exists alone, the control unit 31 assigns a label “EEM” to this region A2. Since the region A3 is a region outside the blood vessel, the control unit 31 assigns a label “Background” to this region A3. Information on the label given by the control unit 31 is temporarily stored in the main storage unit 32.

Although FIG. 10 illustrates a state in which two types of regions of the EEM region and the Lumen region are designated for simplification, an In-Stent region, a plaque region, a thrombus region, a hematoma region, a device region, and the like may be further designated. The control unit 31 determines the overlap between the regions each time the region is designated by the region designation tool 304, and in a case where the plurality of regions overlaps, a plurality of labels corresponding to the respective regions may be assigned, and in a case where the regions do not overlap, a single label corresponding to the region may be assigned.

The segment display field 305 displays information of a region drawn in the image display field 302. The example of FIG. 10 illustrates that the EEM region and the Lumen region are displayed in the image display field 302.

The editing tool 306 is a tool for accepting editing of a region drawn in the image display field 302, and includes a selection button, an edit button, an erase button, an end button, and a color setting field. By using the editing tool 306, with respect to the region already drawn in the image display field 302, it is possible to move, add, and erase points that define the region, and change the color of the region.

When saving of the annotation is selected by the file selection tool 301 after the designation or editing of the region is completed, the control unit 31 stores a data set including the data of the tomographic image (i.e., annotation data) and the data of the label attached to each region in the auxiliary storage unit 35.

In the present embodiment, the annotation is performed by manual work of the user. However, if learning of the learning model MD progresses, the annotation can be performed using the recognition result of the learning model MD. Specifically, the image processing apparatus 3 displays the acquired tomographic image in the image display field 302, performs region recognition using the learning model MD in the background, calculates a plurality of points passing through a boundary line of the recognized region, and plots the points in the image display field 302. Since the image processing apparatus 3 grasps the type of the recognized region, it is possible to automatically assign a label to the region. The image processing apparatus 3 accepts editing of the points plotted in the image display field 302 as necessary, and stores data of a region surrounded by the finally determined points and a label of the region in the auxiliary storage unit 35 as annotation data. Alternatively, annotation support may be performed using a known image processing method.

In the multi-label segmentation task, the learning model MD is generated using the annotation data labeled as described above as the training data, and the EEM region, the Lumen region, the In-Stent region, and the like are simultaneously detected from the newly captured tomographic image using the generated learning model MD. Alternatively, the learning model in which the information regarding the contour of each region is learned may be generated, and the contour of each region may be detected from the newly captured tomographic image using the generated learning model.

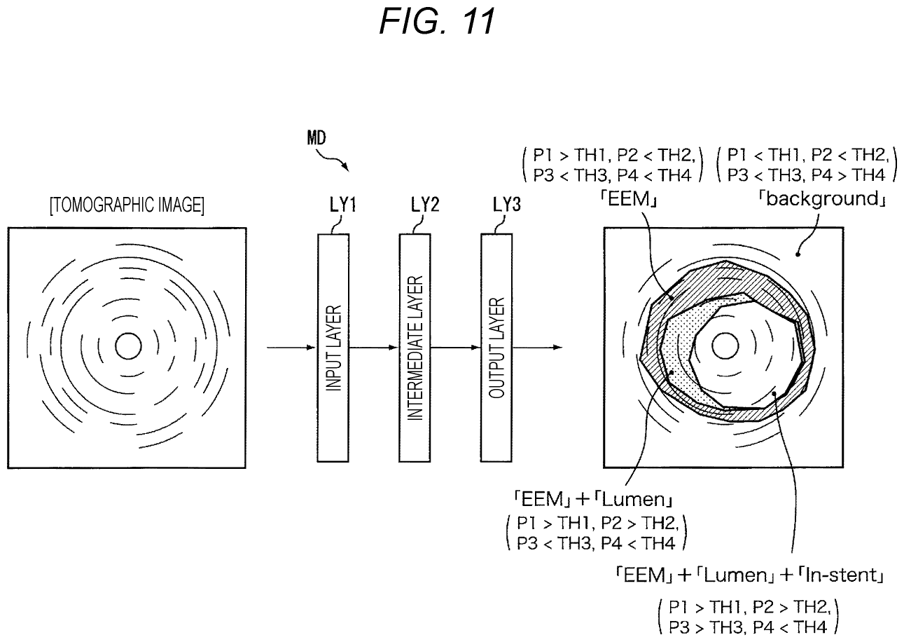

FIG. 11 is a diagram for explaining the configuration of the learning model MD. The learning model MD is a computer learning model that performs semantic segmentation, instance segmentation, and the like. The learning model MD is configured by a neural network such as a convolutional neural network (CNN), and includes an input layer LY1 to which a tomographic image is input, an intermediate layer LY2 that extracts a feature amount of the image, and an output layer LY3 that outputs information of a specific region and a label included in the tomographic image. Note that the tomographic image input to the input layer LY1 may be an image of a frame unit or an image of a plurality of frames. In addition, the tomographic image input to the input layer LY1 may be in an image format described by the XY coordinate system or may be in an image format described by the RO coordinate system. Furthermore, the tomographic image input to the input layer LY1 may be a partial image cut out from the tomographic image or may be the entire tomographic image. Furthermore, the tomographic image input to the input layer LY1 may be an image obtained by combining a plurality of tomographic images. For example, an image obtained by combining a plurality of tomographic images may be an image including line data of more than 360 degrees (i.e., one normal frame), or may be an image in which an image of another frame is put in each of 3ch (RGB layers) of the input image.

The input layer LY1 of the learning model MD includes a plurality of neurons that receives an input of a pixel value of each pixel included in the tomographic image, and passes the input pixel value to the intermediate layer LY2. The intermediate layer LY2 has a configuration in which a convolution layer for convoluting a pixel value of each pixel input to the input layer LY1 and a pooling layer for mapping a pixel value convoluted by the convolution layer are alternately connected, and extracts a feature amount of an image while compressing pixel information of a tomographic image. The intermediate layer LY2 passes the extracted feature amount to the output layer LY3. The output layer LY3 outputs information such as a position and a label of a specific region included in the image.

For example, the output layer LY3 individually calculates, for each pixel (or each region) constituting the tomographic image, the probability P1 that the pixel (or region) corresponds to “EEM”, the probability P2 that corresponds to “Lumen”, the probability P3 that corresponds to “In-Stent”, and the probability P4 that corresponds to “background” by the sigmoid function, and outputs the probabilities P1, P2, P3, and P4. Each of the probabilities P1 to P4 takes a real value between 0 to 1. The control unit 31 compares the magnitude relationship between the probabilities P1 to P4 calculated in the output layer LY3 and the threshold values TH1 to TH4 set to the respective labels “EEM”, “Lumen”, “In-Stent”, and “background”, and determines to which label the target pixel (or region) belongs.

For example, it can be determined that the pixels determined as P1>TH1, P2 21 TH2, P3<TH3, and P4<TH4 belong to “EEM”. In addition, pixels determined as P1>TH1, P2>TH2, P3<TH3, and P4<TH4 can be determined to belong to “EEM” and “Lumen”, and P1>TH1, P2>TH2, P3>TH3, and P4<TH4 can be determined to belong to “EEM”, “Lumen”, and “In-Stent”. Furthermore, pixels determined as P1<TH1, P2<TH2, P3<TH3, and P4>TH4 can be determined to belong to “background”. That is, since the multi-label segmentation task is adopted in the present embodiment, it is possible to correctly recognize the label to which the region belongs for a single region, and correctly recognize each label to which each region belongs for a region in which a plurality of regions overlaps.

Note that the threshold value for each label can be set on a rule basis. In addition, for the overlapping region, the output result of the sigmoid function of all the labels may be used as an input, and the final label may be determined using a learning model learned to output the final determination label.

In the present embodiment, the probabilities P1 to P4 corresponding to “EEM”, “Lumen”, “In-Stent”, and “background” are calculated in the output layer LY3, but the probability corresponding to “plaque”, the probability corresponding to “thrombus”, the probability corresponding to “hematoma”, and the probability corresponding to “device” may be further calculated. Furthermore, the learning model MD may include a neural network other than the CNN, such as SegNet (a method of semantic segmentation), a Single Shot Multibox Detector (SSD), a Spatial Pyramid Pooling Network (SPPnet), a Support Vector Machine (SVM), a Bayesian network, or a regression tree.

Hereinafter, the operation of the image processing apparatus 3 will be described.

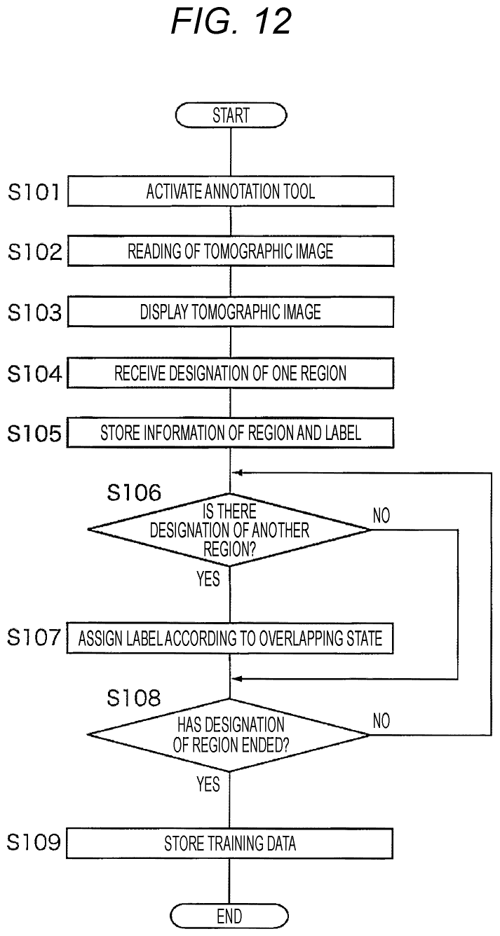

FIG. 12 is a flowchart for explaining an annotation execution procedure according to the present embodiment. In the learning phase, annotation work is performed on the tomographic image of the blood vessel. At this time, the annotation tool AT is activated in the image processing apparatus 3 (S101). When the annotation tool AT is activated, the control unit 31 of the image processing apparatus 3 displays the work screen 300 as illustrated in FIG. 10 on the display apparatus 4.

The control unit 31 reads a tomographic image when a file selection operation is performed through the file selection tool 301 (S102). The tomographic image includes, for example, a plurality of frames. The control unit 31 displays the tomographic image of the frame designated by the frame designation tool 303 in the image display field 302 (S103).

The control unit 31 receives designation of one region for the tomographic image displayed in the image display field 302 (S104). Specifically, after the software button corresponding to one label is selected from the region designation tool 304, a plot of a plurality of points surrounding one region is received in the image display field 302. The control unit 31 temporarily stores the information of the region (for example, a region surrounded by a closed curve) surrounded by the plotted points and the information of the selected label in the main storage unit 32 (S105).

The control unit 31 determines whether designation of another region has been received for the tomographic image displayed in the image display field 302 (S106). Specifically, after the software button corresponding to another label is selected from the region designation tool 304, the control unit 31 determines whether plots of a plurality of points surrounding another region are received in the image display field 302.

When the designation of another region has not been received (S106: NO), the control unit 31 executes the processing of S108 and subsequent steps. When it is determined that the designation of another region has been accepted (S106: YES), the control unit 31 determines the overlapping state of each region and assigns a label to each region according to the overlapping state (S107). For example, in a case where one region designated in S104 is “EEM” and the other region designated in S106 is “Lumen”, a label “EEM” is given to a region where the “EEM” region does not overlap the “Lumen” region, a label “Lumen” is given to a region where the “Lumen” region does not overlap the “EEM” region, and labels “EEM” and “Lumen” are given to a region where the “EEM” region and the “Lumen” region overlap. The same applies to a case where a region belonging to another label is designated or a case where there are three or more designated regions, and the control unit 31 may set the label according to the superimposed state of the plurality of regions.

Next, the control unit 31 determines whether to end the designation of the region (S108). When the saving of the annotation is selected by the file selection tool 301, the control unit 31 determines to end the designation of the region (S108: YES), and stores the data of the tomographic image, the information of each designated region, and the information of the label given to each region in the auxiliary storage unit 35 as training data for generating the learning model MD (S109).

On the other hand, when determining not to end the designation of the region (S108: NO), the control unit 31 returns the process to S106. When the designation of the region is newly received or when the editing of the region is received through the editing tool 306, the control unit 31 executes processing of updating the definition of the region according to the overlapping state of each region or updating the label to be assigned to the region.

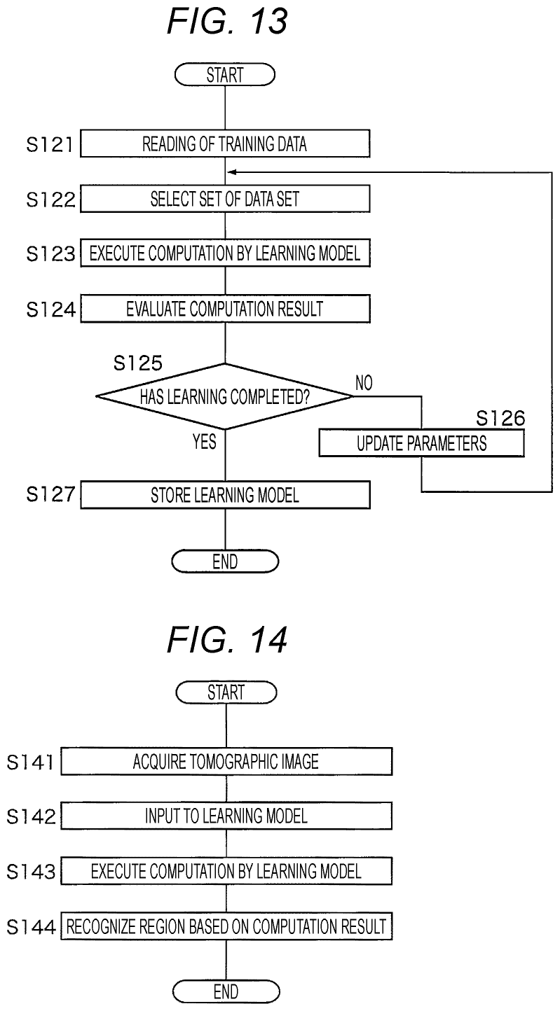

FIG. 13 is a flowchart for explaining a generation procedure of the learning model MD. The control unit 31 of the image processing apparatus 3 reads a learning processing program (not illustrated) from the auxiliary storage unit 35 and executes the following procedure to generate the learning model MD. Note that it is assumed that an initial value is given to the definition information describing the learning model MD before starting learning.

The control unit 31 accesses the auxiliary storage unit 35 and reads training data prepared in advance for generating the learning model MD (S121). The control unit 31 selects a set of data sets (i.e., tomographic images and label data of each region) from the read training data (S122).

The control unit 31 inputs the tomographic image included in the selected training data to the learning model MD and executes computation by the learning model MD (S123). That is, the control unit 31 inputs the pixel value of each pixel included in the tomographic image to the input layer LY1 of the learning model MD, extracts the feature amount of the image while alternately executing the processing of convoluting the input pixel value of each pixel and the processing of mapping the convoluted pixel value in the intermediate layer LY2, and executes the computation of outputting the information such as the position and the label of the specific region included in the image from the output layer LY3.

The control unit 31 acquires a computation result from the learning model MD and evaluates the acquired computation result (S124). For example, the control unit 31 can evaluate the computation result by the learning model MD by comparing information of the region recognized as the computation result with information (or correct answer data) of the region included in the training data.

The control unit 31 determines whether the learning is completed based on the evaluation of the computation result (S125). The control unit 31 calculates the similarity between the information of the region recognized as the computation result of the learning model MD and the information (or correct data) of the region included in the training data, and may determine that the learning is completed when the calculated similarity is equal to or greater than a threshold value.

When determining that the learning is not completed (S125: NO), the control unit 31 sequentially updates the weight coefficient and the bias in each layer of the learning model MD from the output side to the input side of the learning model MD using the backpropagation method (S126). After updating the weight coefficient and the bias of each layer, the control unit 31 returns the processing to S122 and executes the processing from S122 to S125 again.

When it is determined in S125 that the learning is completed (S125: YES), since the trained learning model MD is obtained, the control unit 31 stores the learning model MD in the auxiliary storage unit 35 (S127), and ends the processing according to this flowchart.

FIG. 14 is a flowchart for explaining a region recognition procedure by the learning model MD. The control unit 31 of the image processing apparatus 3 executes the following processing at a timing after generating the learning model MD. The control unit 31 acquires a tomographic image of the blood vessel captured by the intravascular inspection apparatus 101 from the input/output unit 33 (S141).

The control unit 31 inputs the acquired tomographic image to the learning model MD (S142) and executes computation by the learning model MD (S143).

The control unit 31 recognizes a region based on the computation result by the learning model MD (S144). Specifically, the control unit 31 recognizes each region by comparing the probability for each pixel (or each region) output from the output layer LY3 of the learning model MD with a threshold value and determining to which label the pixel (or region) belongs according to the comparison result.

After recognizing each region in S144, the control unit 31 may apply contour correction or a rule-based algorithm to acquire a final recognition result. Here, in the rule-based algorithm, for example, an obvious rule that the lumen region does not exceed the outside of the blood vessel region and that the region such as the blood vessel region or the lumen region and the background region do not overlap can be used.

After the recognition in S144 or the contour correction, the control unit 31 can display the tomographic image with information indicating the recognized regions on the display apparatus 4 so that a medical doctor can make a medical diagnosis.

Note that the output layer of the learning model MD used at the time of region recognition may be different from the output layer at the time of learning. For example, after changing the output layer of the trained learning model MD so as not to recognize a part of the learned region, the control unit 31 may execute the procedures of S141 to S144 described above and perform the recognition processing using the learning model MD.

In the present embodiment, for a single region in which a plurality of regions does not overlap, a label to which the region belongs can be recognized, and for a region in which a plurality of regions overlaps, all labels to which each of the overlapping regions belongs can be recognized. For example, in the case illustrated in FIG. 8, since the lumen region and the stent region overlap, the lumen region cannot be recognized when the stent region is recognized by the conventional single label segmentation task. On the other hand, in the present embodiment, since the multi-label segmentation task is adopted, the lumen region and the stent region can be recognized at a time without separately executing the detection processing of the lumen region.

In the present embodiment, the region is recognized using the multi-label segmentation task. In this task, semantic segmentation learning is performed for each label (i.e., loss is calculated and added for each label) regardless of the channel. That is, since there is a possibility that the regions overlap for each label, the sigmoid function is used as the activation function of the output layer. Alternatively, multi-task single-label segmentation may be implemented. In this case, the neural network for feature extraction may be shared, and semantic segmentation learning may be performed for each channel in parallel (i.e., loss is calculated for each channel). Since there is no overlapping region in the channel, a softmax function can be used as the activation function of the output layer.

In addition, as an auxiliary task for improving accuracy, a lesion angle task may be introduced, and the auxiliary task may be separated and used at the time of inference.

### Second Embodiment

In the first embodiment, the IVUS image and the OCT image are not

distinguished, and the tomographic image of the blood vessel is input to the learning model MD to recognize a plurality of regions included in the tomographic image. In the second embodiment, a configuration for performing region recognition using a first learning model that outputs information of a plurality of regions included in an IVUS image in response to an input of the IVUS image and a second learning model that outputs information of a plurality of regions included in an OCT image in response to an input of the OCT image will be described.

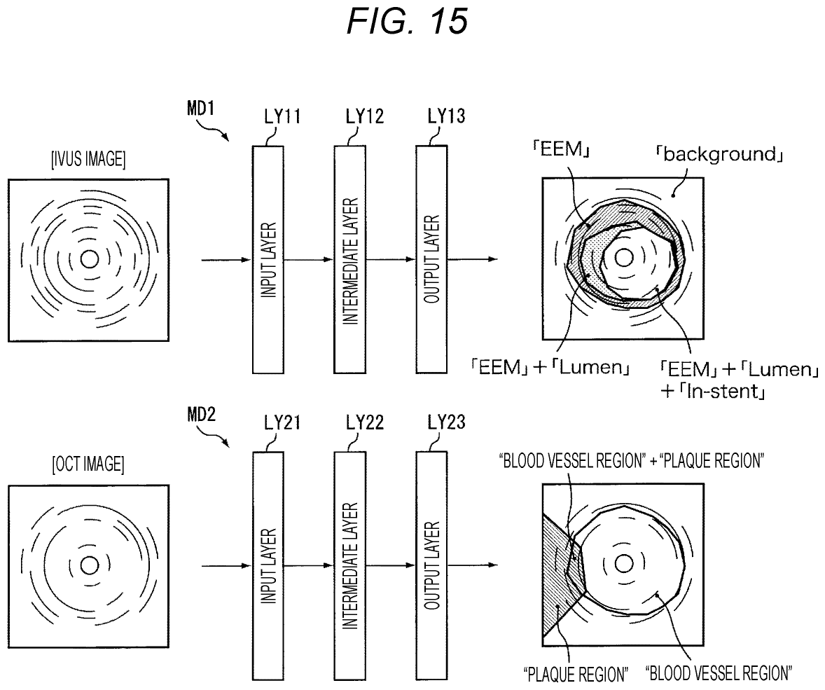

FIG. 15 is a diagram for explaining the configurations of the first learning model MD1 and the second learning model MD2 in the second embodiment. The first learning model MD1 and the second learning model MD2 are learning models that perform semantic segmentation, instance segmentation, and the like, similarly to the learning model MD of the first embodiment, and are configured by a neural network such as CNN.

The first learning model MD1 is a computer learning model that outputs information of a plurality of regions included in an IVUS image in response to an input of the IVUS image, and includes an input layer LY11 to which the IVUS image is input, an intermediate layer LY12 that extracts a feature amount of the image, and an output layer LY13 that outputs information of a specific region and a label included in the IVUS image. The input of the IVUS image may include information of one frame or information of a plurality of frames.

In a case where an IVUS image is input, the first learning model MD1 is learned to output a probability that the pixel (or region) corresponds to “EEM”, “Lumen”, “In-Stent”, and “background” for each pixel (or region) constituting the IVUS image. In the present embodiment, “background” represents a background region with respect to the EEM region, but “background” may be set for each of the EEM region, the Lumen region, and the In-Stent region. Since the annotation method in generating the training data and the learning procedure using the training data are similar to those in the first embodiment, the description thereof will be omitted.

The second learning model MD2 is a computer learning model that outputs information of a plurality of regions included in the OCT image in response to an input of the OCT image, and includes an input layer LY21 to which the OCT image is input, an intermediate layer LY22 that extracts a feature amount of the image, and an output layer LY23 that outputs information of a specific region and a label included in the OCT image.

In a case where the OCT image is input, the second learning model MD2 is learned to output the probability that the pixel (or region) corresponds to the “blood vessel region”, the “plaque region”, the “thrombus region”, and the “hematoma region” for each pixel (or region) constituting the OCT image. Note that the annotation method in generating the training data and the learning procedure using the training data are similar to those in the first embodiment, and thus the description thereof will be omitted.

When performing region recognition using the trained first learning model MD1 and second learning model MD2, the image processing apparatus 3 according to the second embodiment inputs an IVUS image to the first learning model MD1 and an OCT image to the second learning model MD2 among tomographic images captured by the intravascular inspection apparatus 101. The control unit 31 of the image processing apparatus 3 performs computation by the first learning model MD1, and recognizes regions corresponding to “EEM”, “Lumen”, “In-Stent”, and “background” based on information output from the learning model MD1. Furthermore, the control unit 31 of the image processing apparatus 3 performs computation using the second learning model MD2, and recognizes regions corresponding to “blood vessel region”, “plaque region”, “thrombus region”, and “hematoma region” based on information output from the learning model MD2.

In the second embodiment, since the blood vessel region, the plaque region, the thrombus region, and the hematoma region are recognized using the OCT image having higher resolution and definition than the IVUS image, it is possible to accurately recognize each region.

Note that, in the second embodiment, each region is recognized using two types of learning models of the first learning model MD1 to which the IVUS image is input and the second learning model MD2 to which the OCT image is input. However, each region may be recognized using a learning model in which the IVUS image and the OCT image are simply pasted together to form one image and segmentation is performed from the one image. In addition, the IVUS image and the OCT image may be combined as different channels, and each region may be recognized using a learning model that performs segmentation from the combined image.

Furthermore, in the first and second embodiments, the description has been given using an intravascular image such as an IVUS image or an OCT image, but the present invention can be applied to an image including other blood vessel tomographic images such as a body surface echo image.

It should be understood that the embodiments disclosed herein are illustrative in all respects and are not restrictive. The scope of the present invention is defined not by the meanings described above but by the claims, and is intended to include meanings equivalent to the claims and all modifications within the scope.

What is claimed is:

1. An image diagnostic system comprising:
a catheter insertable into a blood vessel;
a memory that stores a program; and
a processor configured to execute the program to:
control the catheter to acquire a tomographic image of a blood vessel,
input the acquired image into a computer model to generate information that indicates a plurality of predetermined regions of the blood vessel in the image, the information further indicating for each of the predetermined regions whether it overlaps another region,
the computer model having been trained with a plurality of tomographic images of blood vessels and a plurality of information each specifying the predetermined regions that can overlap in a corresponding one of the tomograph images, and
using the generated information, determine the predetermined regions of the blood vessel in the acquired image, and
output information indicating the determined regions.

2. The image diagnostic system according to claim 1, wherein
the processor executes the program further to:
determine a closed area in the acquired image in which two or more predetermined regions overlap,
assign two or more labels corresponding to said two or more predetermined regions to the closed area, and
store, in the memory, information that associates the closed area with the assigned labels.

3. The image diagnostic system according to claim 1, wherein
the predetermined regions include at least two of a region inside a stent, a region of a lumen, and a region inside an external elastic membrane.

4. The image diagnostic system according to claim 3, wherein
the predetermined regions further include at least one of a plaque region, a thrombus region, a hematoma region, and a medical device region.

5. The image diagnostic system according to claim 1, further comprising:
a display, wherein
the processor executes the program further to control the display to display the information indicating the determined regions.

6. The image diagnostic system according to claim 1, wherein
the generated information indicates, for each of pixels of the input image, probabilities that the pixel corresponds to the predetermined regions.

7. The image diagnostic system according to claim 6, wherein
the processor executes the program to compare the probabilities with thresholds to determine the predetermined regions.

8. The image diagnostic system according to claim 1, wherein
the catheter includes:
a first sensor configured to transmit ultrasonic waves and receive the waves reflected by the blood vessel while the catheter is inserted in the blood vessel, and
a second sensor configured to emit light and receive the light reflected by the blood vessel while the catheter is inserted in the blood vessel, and
the processor executes the program to:
generate an ultrasonic tomographic image of the blood vessel based on the reflected waves received by the first sensor and an optical coherence tomographic image of the blood vessel based on the reflected light received by the second sensor, and
input the generated images into the computer model to generate the information.

9. The image diagnostic system according to claim 1, further comprising:
an angiography apparatus configured to generate an angiographic image of the blood vessel, wherein
the catheter includes a marker that can be imaged by the angiography apparatus.

10. An image diagnostic system comprising:
a catheter insertable into a blood vessel;
a display;
a memory that stores a program; and
a processor configured to execute the program to:
control the catheter to acquire a tomographic image of a blood vessel, control the display to display a screen showing the acquired image and one or more graphical user interface (GUI) components by which a plurality of predetermined regions of the blood vessel can be specified on the image such that each predetermined region can overlap with another predetermined region, and
after the predetermined regions are specified through the GUI components, train a computer model with the image and information indicating the specified regions so that the computer model outputs, upon input of tomographic images, information indicating the predetermined regions of blood vessels in the images and for each of the predetermined regions whether it overlaps another region.

11. The image diagnostic system according to claim 10, wherein
the processor executes the program to assign one or more labels each indicating one of the predetermined regions to a closed area in the tomographic image.

12. The image diagnostic system according to claim 11, further comprising:
an input device, wherein
the processor executes the program to specify the closed area upon input of a designation thereof on the displayed image through the input device.

13. The image diagnostic system according to claim 11, wherein
the GUI components include a plurality of buttons corresponding to the labels.

14. The image diagnostic system according to claim 11, wherein
the labels indicate at least two of a stent, a lumen, and an external elastic membrane.

15. The image diagnostic system according to claim 14, wherein
the labels further indicate at least one of a plaque, a thrombus, a hematoma, and a medical device.

16. A method performed by an image diagnostic system that includes a catheter insertable into a blood vessel, the method comprising:
acquiring a tomographic image of a blood vessel using the catheter;
inputting the acquired image into a computer model to generate information that indicates a plurality of predetermined regions of the blood vessel in the image, the information further indicating for each of the predetermined regions whether it overlaps another region,
the computer model having been trained with a plurality of tomographic images of blood vessels and a plurality of information each specifying the predetermined regions that can overlap in a corresponding one of the tomograph images; and
using the generated information, determining the predetermined regions of the blood vessel in the acquired image; and
outputting information indicating the determined regions.

17. The method according to claim 16, further comprising:
determining a closed area in the acquired image in which two or more predetermined regions overlap,
assigning two or more labels corresponding to said two or more predetermined regions to the closed area, and
storing information that associates the closed area with the assigned labels.

18. The method according to claim 16, wherein
the predetermined regions include at least two of a region inside of a stent, a region of a lumen, and a region inside an external elastic membrane.

19. The method according to claim 18, wherein
the predetermined regions further include at least one of a plaque region, a thrombus region, a hematoma region, and a medical device region.

20. The method according to claim 16, further comprising:
displaying the information indicating the determined regions.
